(19) United States
(12) Patent Application Publication
(10) Pub. No.: US 2001/0036485 A1
(43) Pub. Date: Nov. 1, 2001

(54) Herbal composition and method for promoting emotional well-being

(76) Inventors: Thomas Newmark, St. Louis, MO (US); Paul Schulick, Brattleboro, VT (US)

Correspondence Address: Mary A. Montebello, PATTON BOGGS LLP, 2550 M Street, NW, Washington, DC, 20037, US

(21) Appl. No.: 09/816,746

(22) Filed: Mar. 26, 2001

Related U.S. Application Data
(60) Provisional application No. 60/191,739, filed on Mar. 24, 2000.
(60) Provisional application No. 60/199,821, filed on Apr. 26, 2000.

(51) Int. Cl. A61K 35/78

(52) U.S. Cl. 424/730

(57) ABSTRACT

An improved St. John's Wort-based herbal composition for promoting emotional health in animals, preferably humans, contains a St. John's Wort supercritical extract composed of highly stable hyperforins and rich in essential oil and other components typically found in St. John's Wort extracts. The hyperforin-containing extract is obtained by a supercritical extraction process involving subjecting the St. John's Wort herb to high pressure extraction using compressed gas, most preferably compressed carbon dioxide gas. By using this particular supercritical extraction process to obtain the hyperforin-containing extract, the hyperforins in the extract will be more stable than in extracts retrieved by conventional extraction processes such as processes using water and alcohol. Methods for using the herbal composition to promote emotional health in animals, preferably humans, involve orally administering the composition.

Priority of this Application is based on U.S. Provisional Application No. 60/191,739 (filed Mar. 24, 2000) and U.S. Provisional Application No. 60/199,821 (filed Apr. 26, 2000).

### BACKGROUND OF THE INVENTION

The present invention relates to herbal compositions. More particularly, this invention relates to a St. John's Wort-based herbal composition capable of promoting emotional well being in animals, particularly humans. In addition, this invention relates to a method of using such composition to promote emotional well-being in animals, preferably humans.

St. John's Wort (Hypericum perforatum) is widely recognized for its ability to safely and naturally support positive emotional health. Scientists have extensively studied St. John's Wort to determine which of its constituents has the ability to modulate or balance brain chemistry. Scientists have determined that a class of phytonutrients called hyperforins are most responsible for St. John's Wort's beneficial effects.

Hyperforins are the major component of the oily extract of St. John's Wort and is a component of the hydroalcoholic extract of the herb as well. However, it has been found that in the oily and hydroalcoholic extracts, hyperforins tend to be unstable.

Thus, a primary object of this invention is to provide an herbal composition for promoting emotional well-being in animals, preferably humans, wherein the composition contains a St. John's Wort extract composed of highly stable hyperforins.

A further object of this invention is to provide a method for promoting emotional well-being in animals, preferably humans, involving orally administering the aforementioned herbal composition to an animal (preferably a human) in need of enhanced emotional well-being.

These and other objects are achieved in the present invention.

### SUMMARY OF THE INVENTION

The present invention provides an improved St. John's Wort-based herbal composition for promoting emotional well-being in animals, preferably humans. Specifically, the composition of this invention contains a therapeutically effective amount of a supercritical extract of St. John's Wort, wherein the extract contains highly stable hyperforins. The hyperforins-containing extract used in the present invention is obtained by a supercritical extraction process involving subjecting the St. John's Wort herb to high pressure extraction using compressed gas, most preferably compressed carbon dioxide gas. The present invention is based on the discovery that the hyperforins in an extract obtained using this particular supercritical extraction process will be more stable than the hyperforins in extracts retrieved by conventional extraction processes such as processes using water and alcohol.

A further benefit provided by the present invention is that the supercritical extract used in the composition not only contains highly stable hyperforins but also contains other components found in St. John's Wort extracts. Non-limiting examples of such components include, e.g., essential oils, waxes and resins. Thus, the extract used in this invention is a “total” or “full-spectrum” extract in that it contains a broad spectrum of St. John's Wort lipophilic components, not just one component.

The present invention also provides a method of promoting emotional health in animals, preferably humans, involving orally administering an effective amount of the herbal composition of this invention. In the most preferred embodiments, the orally administered herbal composition is in the form of one or more soft gel capsules.

### DETAILED DESCRIPTION OF THE INVENTION

As stated hereinabove, the present invention provides an improved orally administered herbal composition for promoting emotional well-being in animals, preferably humans. The composition of this invention contains an effective amount of a supercritical extract of St. John's Wort, wherein the extract contains highly stable hyperforins. As used herein, the term “effective amount” with respect to the amount of the supercritical extract administered to an animal, preferably a human, is that amount which is effective in enhancing the emotional well-being of said animal.

The hyperforins-containing supercritical extract used in the composition of this invention is highly stable because of the particular extraction method used to obtain the extract from the St. John's Wort herb. The hyperforins-containing extract is obtained by subjecting the St. John's Wort herb to a high pressure extraction process with compressed gas, most preferably compressed carbon dioxide.

Specifically, the supercritical extraction process used to obtain the stable hyperforins-containing extract used in the present invention involves direct extraction of the St. John's Wort herb (preferably, the flowers of the herb) with a compressed gas as extraction agent. Suitable gases for use as the extraction agent herein are any of those gases which has solvent capacity, that is, a gas which when heated to a temperature exceeding its critical temperature point and pressurized to a pressure exceeding the gas's critical pressure point will enter a fluidized (supercritical non-liquid) state. The most preferred compressed gas for use in the present invention is compressed carbon dioxide.

The high pressure extraction process is preferably carried out at a temperature of from above about 31° C. up to about 80° C, more preferably from about 35° C. to about 50° C, and a pressure of from about 75 bar to about 500 bar, more preferably from about 100 bar to about 250 bar. The amount of compressed gas used for the extraction preferably ranges from about 5 to about 100 grams, more preferably from about 10 to about 40 grams, of the gas per gram of St. John's Wort subjected to the extraction process.

After the supercritical extraction process, the extracted material is separated from the extraction agent by an extraction separator included in the closed gas circulation. Thus, the separated gas is recaptured for reuse.

Typically, the St. John's Wort extract obtained using the high pressure extraction method described above will have a hyperforins content of from about 20% to about 50% by weight.

In addition to stable hyperforins, the supercritical extract used in the present invention further contains other useful components typically found in St. John's Wort extracts, non-limiting examples of such components including monosesquiterpenes, di-and triterpenes, other phloglucinols, carotenoids, phytosterols, fatty acids, cuticular waxes, xanthones and refines.

The therapeutic dosage of the composition of this invention preferably ranges from about 270 mg to about 650 mg of the total extract on a daily basis, which corresponds to about 27 mg to about 65 mg of hyperforins. The therapeutic dosage of the composition can be taken in a single daily administration or it can be divided into several unit dosages to be taken over the course of a day. A unit dosage will depend upon many factors including age, size, and condition of the individual being treated and the number of times the unit will be taken in a single day. In any event, the entire daily dosage will be that which is physiologically acceptable to an animal (preferably a human) and can be administered daily over a prolonged period of time.

As stated above, the herbal composition of this invention is formulated for oral administration. Thus, the composition may be in the form of tablets, capsules, liquids, and the like.

Compositions for use in tablet form will include the herbal extract in admixure with pharmaceutically acceptable inert excipients. These excipients may be, for example, inert diluents such as calcium carbonate, sodium chloride, lactose, calcium phosphate, sodium phosphate, and the like; granulating and disintegrating agents, e.g., potato starch, alginic acid, etc.; binding agents, e.g., starch, gelatin, or acacia, etc.; and lubricating agents, e.g., magnesium stearate, stearic acid or talc. Other pharmaceutically acceptable excipients can be colorants, flavoring agents, plasticizers, humectants, etc. The tablets may be uncoated or they may be coated by known techniques.

Alternatively, the herbal composition may be delivered in a soft gel capsule by mixing the extract with an oil medium such as peanut oil, liquid paraffin, or olive oil and enclosing it within the capsule or gel capsule.

The composition may also be administered as an oral liquid by suspending the active ingredient in an aqueous solution in admixture with a dispersing or wetting agent, suspending agent and one or more preservatives. Suitable dispersing or wetting agents include, e.g., naturally occurring phosphatides (e.g., lecithin), or condensation products of ethylene oxide, fatty acids, long chain aliphatic acids, or a partial ester derived from fatty acids and a hexitol or hexitol anhydrides, e.g., polyoxyethylene stearate, polyoxyethylene sorbitol monooleate, polyoxyethylene sorbitan monooleate, etc. Suitable suspending agents include, e.g., sodium carboxymethylcellulose, methyl cellulose, sodium alginate, etc.

The present invention is also directed to a method of promoting emotional well being in an animal, preferaby a human, involving orally administering an effective amount of the herbal composition of this invention to said animal for an effective period of time. The terms “effective amount” and “effective period of time” with respect to the amount and duration of administration of the herbal composition is that amount and time period which is sufficient to promote a sense of emotional well-being. This amount will tend to vary from individual to individual. Persons of ordinary skill in the art can easily determine optimum dosages, dosing methodologies, and repetition rates. Generally, as mentioned hereinabove, the therapeutic dosage of the composition of this invention preferably ranges from about 270 mg to about 650 mg of the total extract on a daily basis, which corresponds to about 27 mg to about 65 mg of hyperforins.

The composition of this invention is preferably orally administered on a daily basis for a period of at least 4 weeks. If taken on a non-daily basis, the effective period of time will generally be longer, e.g., at least 6 weeks.

The herbal composition is preferably in the form of one or more soft gel capsules. In its most preferred embodiment, the herbal composition of this invention is in the form of a single soft gel capsule containing about 270 mg of the St. John's Wort supercritical extract (taken from the flower of the herb) standardized to a 10% hyperforin content (i.e., 27 mg of hyperforins). Preferably, the composition will further contain olive oil (certified organic) and yellow beeswax. The capsule portion of the soft gel capsules preferably contains gelatin, vegetable glycerine, purified water and carob.

For oral administration of the above-recited formulation, one soft gel capsule (which constitutes one serving of the composition of this invention) is preferably taken daily, with 8 ounces of water or other liquid.

What is claimed is:

1. An orally administered St. John's Wort-based herbal composition for promoting emotional well-being in animals, comprising a therapeutically effective amount of a supercritical extract of a St. John's Wort herb, wherein the supercritical extract comprises highly stable hyperforins, further wherein the hyperforins-containing supercritical extract is prepared by a method comprising subjecting said herb to high pressure extraction using compressed gas as an extraction agent.

2. A composition according to claim 1, wherein, in the method for preparing the hyperforins-containing supercritical extract, the compressed gas is compressed carbon dioxide.

3. A composition according to claim 1, wherein, in the method for preparing the hyperforins-containing supercritical extract, the high pressure extraction is carried out at a temperature of from about 31° C. to about 80° C.

4. A composition according to claim 1, wherein, in the method for preparing the hyperforins-containing supercritical extract, the high pressure extraction is carried out at a pressure of from about 75 bar to about 500 bar.

5. A composition according to claim 1, wherein, in the method for preparing the hyperforins-containing supercritical extract, the amount of compressed gas ranges from about 5 to about 100 grams per 1 gram of the St. John's Wort herb subjected to the supercritical extraction.

6. A composition according to claim 1, wherein the supercritical extract comprises from about 20% to about 50% by weight of said hyperforins.

7. A composition according to claim 1, wherein the supercritical extract further comprises one or more of monosesquiterpenes, diterpenes, triterpenes, phloglucinols, carotenoids, phytosterols, fatty acids, cuticular waxes, xanthones and refines.

8. A composition according to claim 1, wherein: in the method for preparing the hyperforins-containing supercritical extract, the compressed gas is compressed carbon dioxide; the amount of the compressed carbon dioxide is from about 5 to about 100 grams per 1 gram of the St. John's wort herb subjected to the supercritical extraction; and the high pressure extraction is carried out at a temperature of from about 31° C. to about 80° C. and a pressure of from about 75 bar to about 500 bar; farther wherein the supercritical extract comprises from about 20% to about 50% by weight of said hyperforins:

9. A method for promoting emotional well-being in animals, comprising orally administering to an animal for an effective period of time an effective amount of a St. John's Wort-based herbal composition comprising a therapeutically effective amount of a supercritical extract of a St. John's Wort herb, wherein the supercritical extract comprises highly stable hyperforins, further wherein the hyperforins-containing supercritical extract is prepared by a method comprising subjecting said herb to high pressure extraction using compressed gas as an extraction agent.

10. A method according to claim 9, wherein, in the method for preparing the hyperforins-containing supercritical extract, the compressed gas is compressed carbon dioxide.

11. A method according to claim 9, wherein, in the method for preparing the hyperforins-containing supercritical extract, the high pressure extraction is carried out at a temperature of from about 31° C. to about 80° C.

12. A method according to claim 9, wherein, in the method for preparing the hyperforins-containing supercritical extract, the high pressure extraction is carried out at a pressure of from about 75 bar to about 500 bar.

13. A method according to claim 9, wherein, in the method for preparing the hyperforins-containing supercritical extract, the amount of compressed gas ranges from about 5 to about 100 grams per 1 gram of the St. John's Wort herb subjected to the supercritical extraction.

14. A method according to claim 9, wherein, in the orally administered composition, the supercritical extract comprises from about 20% to about 50% by weight of said hyperforins.

15. A method according to claim 9, wherein, in the orally administered composition, the supercritical extract further comprises one or more of monosesquiterpenes, diterpenes, triterpenes, phloglucinols, carotenoids, phytosterols, fatty acids, cuticular waxes, xanthones and refines.

16. A method according to claim 9, wherein, in the method for preparing the hyperforins-containing supercritical extract, the compressed gas is compressed carbon dioxide; the amount of the compressed carbon dioxide is from about 5 to about 100 grams per 1 gram of the St. John's Wort herb subjected to the supercritical extraction; and the high pressure extraction is carried out at a temperature of from about 31° C. to about 80° C. and a pressure of from about 75 bar to about 500 bar; further wherein the supercritical extract comprises from about 20% to about 50% by weight of said hyperforins.

17. A method according to claim 9, wherein the animal is human.

18. A method according to claim 17, wherein the composition is orally administered to said human on a daily basis.

19. A method according to claim 18, wherein the amount of the composition orally administered to said human is such as to provide from about 270 to about 650 mg of the hyperforins-containing supercritical extract to said human on a daily basis.

20. A method according to claim 18, wherein the amount of the composition orally administered to said human is such as to provide from about 27 to about 65 mg of the highly stable hyperforins to said human on a daily basis.

21. A method according to claim 18, wherein the composition is orally administered to said human for a period of at least about 4 weeks.
